(19) United States
(12) Patent Application Publication
(10) Pub. No.: US 2010/0061506 A1
(43) Pub. Date: Mar. 11, 2010

(54) RADIATION-AND-DETECTION SYSTEM

(75) Inventor: Thomas Koehler, Norderstedt (DE)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELECTRONICS N. V., Eindhoven (NL)

(21) Appl. No.: 12/516,356

(22) PCT Filed: Nov. 21, 2007
(86) PCT No.: PCT/IB07/54726

§ 371 Date: May 27, 2009

(30) Foreign Application Priority Data
Nov. 28, 2006 (EP) 06124941.3

(51) Int. Cl. G01D 18/00; A61B 6/03

(52) U.S. Cl. 378/19; 378/207

(57) ABSTRACT

The invention relates to radiation-and-detection system comprising a radiation unit (2) for alternately illuminating a detection unit (6) with radiation of a first emanating region (15) at first time intervals and with radiation of a second emanating region (16) at second time intervals, wherein first detection values are detected at the first time intervals and second detection values are detected at the second time intervals. The radiation-and-detection system is calibrated with respect to the influence of the radiation of the second emanating region (16) to the first detection values and with respect to the influence of the radiation of the first emanating region (15) to the second detection values.

### FIELD OF THE INVENTION

The present invention relates to a radiation-and-detection system and a radiation-and-detection method.

### BACKGROUND OF THE INVENTION

Radiation-and-detection systems are known, which comprise a radiation unit and a detection unit, wherein the radiation unit has a first emanating region and a second emanating region for alternately illuminating the detection unit with radiation of the first emanating region at first time intervals and with radiation of the second emanating region at second time intervals. Such a radiation-and-detection system is, for example, a computed tomography system (CT system), which comprises an X-ray stereo tube having two focal spots, wherein a region of interest is alternately illuminated by the radiation emanating from the two focal spots. The radiation of the two focal spots after having traversed the region of interest is detected by the detection unit. The detection unit generates detection values depending on the radiation, and an image of the region of interest is reconstructed using the generated detection values. Radiation-and-detection systems comprising the above mentioned radiation unit have the drawback that the detection values are faulty due to the use of the first and second emanating regions yielding reconstructed images comprising artifacts.

### SUMMARY OF THE INVENTION

It is an object of the present invention to provide a radiation-and-detection system comprising a radiation unit having a first emanating region and a second emanating region for alternately illuminating the detection unit with radiation of the first emanating region at first time intervals and with radiation of the second emanating region at second time intervals, wherein the degree of faultiness of the detection values is reduced.

In a first aspect of the present invention a radiation-and-detection system is presented that comprises:

a radiation unit having a first emanating region and a second emanating region for alternately illuminating the detection unit with radiation of the first emanating region at first time intervals and with radiation of the second emanating region at second time intervals,

a detection unit for detecting values depending on the radiation, the detection unit being adapted for detecting first detection values at the first time intervals and second detection values at the second time intervals,

a calibration unit

for calibrating the radiation-and-detection system with respect to the influence of the radiation of the second emanating region to the first detection values, the calibration unit being adapted for determining calibration values, which depend on the influence of the radiation of the second emanating region to the first detection values, for correcting the first detection values, and

for calibrating the radiation-and-detection system with respect to the influence of the radiation of the first emanating region to the second detection values, the calibration unit being adapted for determining calibration values, which depend on the influence of the radiation of the first emanating region to the second detection values, for correcting the second detection values.

The invention is based on the idea that, in the above mentioned prior art, the faultiness of the first detection values is caused by the influence of the radiation of the second emanating region to the first detection values. For example, the afterglow of the detection unit caused by the radiation of the second emanating region influences the first detection signals. Furthermore, the second emanating region could emanate some residual radiation also at the first time intervals, for example, because of physical limitations in the switching time between the first and second emanating regions. In the same way, the faultiness of the second detection values is caused by the influence of the radiation of the first emanating region. A calibration for correcting the first and second detection values with respect to these influences decreases the degree of faultiness of the first and second detection values.

It is preferred that the calibration unit is adapted for using at least one of the first and second detection values to determine the calibration values for correcting the first detection values, and that the calibration unit is adapted for using at least one of the first and second detection values to determine the calibration values for correcting the second detection values. Since the detection values of the radiation-and-detection system itself are used for determining the calibration values and not, for example, detection values measured in a laboratory, the calibration values can be determined with a high accuracy.

It is further preferred that the radiation-and-detection system comprises a prevention unit for preventing the radiation of one of the first and the second emanating regions from being detected by the detection unit. This allows determining the calibration values for correction the first detection values using at least one of the first and second detection values, which have been acquired while the radiation of the first emanating region has been prevented from being detected by the detection unit. Furthermore, this allows to determine the calibration values for correcting the second detection values using at least one of the first and second detection values, which have been acquired while the radiation of the second emanating region has been prevented from being detected by the detection unit. Therefore, this allows determining the influence of the radiation of one of the first and second emanating regions, wherein this determination is not effected by the radiation of the other of the first and second emanating regions. This improves the quality of the calibration values and, thus, of the corrected detection values and of images, which might be reconstructed by using these corrected detection values.

The prevention unit is preferentially a blocking element for blocking radiation. This blocking element is, for example, a collimator or a metal plate, in particular, of a collimator. This allows preventing the radiation of one of the first and second emanating regions from being detected by the detection unit, while the radiation unit can still emanate radiation from both emanating region alternatively. Therefore, during calibration the radiation unit can operate in the same mode as in an image generation mode, which is used for generating detection values for reconstructing an image of a region of interest, after the radiation-and-detection system has been calibrated, i.e. after the calibration values have been determined. Since the mode of operation of the radiation unit can be the same during the calibration mode and during the image generation mode, the determination of the calibration values is based on real image generation conditions, which improves the quality of the calibration values and, therefore, of the corrected detection values and of an image, which might be reconstructed using these corrected detection values. For example, if the radiation unit is an X-ray stereo tube having two focal spots, wherein the radiation of these two focal spots illuminates a region of interest alternately, during calibration the X-ray stereo tube can alternately radiate, wherein the radiation of only one focal spot reaches the detection unit.

It is further preferred

that, for determining calibrating values for correcting the first detection values,

the prevention unit is adapted for preventing the detection of the radiation of the first emanating region by the detection unit,

the detection unit is adapted for detecting first and second detection values, and

the calibration unit is adapted for determining calibration values for correcting the first detection values by using a ratio of successive first and second detection values, and that, for determining calibrating values for correcting the second detection values,

the prevention unit is adapted for preventing the detection of the radiation of the second emanating region by the detection unit,

the detection unit is adapted for detecting first and second detection values, and

the calibration unit is adapted for determining calibration values for correcting the second detection values by using a ratio of successive first and second detection values. This allows to determine high quality calibration values.

The calibration unit is preferentially adapted for determining calibration values which depend on an afterglow value of the detection unit. Since the afterglow generally influences the detection values, the use of an afterglow value of the detection unit for determining the calibration values further improves the quality of the corrected detection values and, therefore, of an image, which might be reconstructed by using the corrected detection values.

It is further preferred that the calibration unit is adapted for determining calibration values which depend on residual radiation of an emanating region. Since the residual radiation generally influences the detection values, the consideration of the residual radiation for determining the calibration values further improves the quality of the corrected detection values and, therefore, of an image, which might be reconstructed by using the corrected detection values.

The radiation-and-detection system preferentially further comprises a correction unit, which is adapted for correcting the first and second detection values using the calibration values. The correction unit allows to provide corrected detection values, which can be used for reconstruction an image of a region of interest.

The correction unit is preferentially adapted for correcting the first and second detection values using following steps:

modeling the first and second detection values as a convolution of corrected first and second values with a kernel, which depends on the calibration values,

recalculating the corrected first and second detection values by inverting the convolution.

It is further preferred that the correction unit is adapted for correcting the first and second detection values using following steps:

modeling the first and second detection values as a matrix equation, wherein the first and second detection values are connected to corrected first and second detection values by a matrix, which depends on the calibration values,

recalculating the corrected first and second detection values by inverting the matrix equation. This model and recalculation allows determining corrected detection values with an improved accuracy.

The radiation-and-detection system preferentially comprises a reconstruction unit for reconstructing an image of a region of interest located between the radiation unit and the detection unit using the corrected first and second detection values. Since the reconstruction unit reconstructs an image of the region of interest by using the corrected detection values, a high quality image is generated.

The radiation-and-detection system is preferentially a computed tomography system, wherein the radiation unit is preferentially an X-ray stereo tube. The X-ray stereo tube comprises two focal spots, wherein radiation emanating from these two focal spots illuminates the detection unit alternately. In theory, the detection unit is in the first time interval illuminated by radiation emanating from a first focal spot only, and the detection unit is illuminated in the second time intervals by radiation emanating from a second focal spot only. But, in practice, generally, if the detection unit is supposed to be illuminated by radiation emanating from one of the focal spots, a residual X-ray flux of the other of the focal spots is still present. This residual X-ray flux and/or the afterglow of the detection unit influence the detection values. This influence is corrected by determining the calibration values and by using the determined calibration values for correcting the detection values. The use of these corrected detection values by the computed tomography system improves the quality of images reconstructed by the computed tomography system.

It is a further object of the invention to provide a radiation-and-detection system having a radiation unit and a detection unit, wherein the radiation unit has an emanating region for intermittently illuminating the detection unit and wherein the quality of detection values generated by the detection unit is improved.

In an aspect of the invention a radiation-and-detection system is presented, wherein the radiation-and-detection system comprises

a radiation unit having an emanating region for intermittently illuminating a detection unit at first time intervals,

a detection unit for detecting detection values depending on the radiation at second time intervals located between the first time intervals,

a calibration unit for calibrating the radiation-and-detection system with respect to the influence of the radiation emanated at the first time intervals to the detection values detected at the second time intervals, the calibration unit being adapted for determining calibration values, which depend on the influence of the radiation emanated at the first time intervals to the detection values detected at the second time intervals, for correcting the first detection values and second detection values.

In a further aspect of the invention an image generation system for generating an image of a region of interest is presented, the image generation system being provided with

first detection values being detected at first time intervals and second detection values being detected at second time intervals, wherein a radiation unit has alternately illuminated the detection unit with radiation from a first emanating region at first time intervals and a second emanating region at second time intervals,

calibration values, which depend on the influence of the radiation of the second emanating region to the first detection values, for correcting the first detection values,

calibration values, which depend on the influence of the radiation of the first emanating region to the second detection values, for correcting the second detection values, the image generation unit comprising:

a correction unit being adapted for correcting the first and second detection values using the calibration values,

a reconstruction unit for reconstructing an image of a region of interest located between the radiation unit and the detection unit using the corrected first and second detection values.

In a further aspect of the invention a radiation-and-detection method for calibrating a radiation-and-detection system is provided, wherein the radiation-and-detection method comprises following steps:

alternately illuminating a detection unit with radiation of a first emanating region at first time intervals and with radiation of a second emanating region at second time intervals,

detecting values depending on the radiation, wherein first detection values are detected at the first time intervals and second detection values are detected at the second time intervals,

calibrating the radiation-and-detection system with respect to the influence of the radiation of the second emanating region to the first detection values, wherein calibration values are determined, which depend on the influence of the radiation of the second emanating region to the first detection values, for correcting the first detection values, and

calibrating the radiation-and-detection system with respect to the influence of the radiation of the first emanating region to the second detection values, wherein calibration values are determined, which depend on the influence of the radiation of the first emanating region to the second detection values, for correcting the second detection values.

In a further aspect of the invention a computer program for calibrating a radiation-and-detection system is presented, the computer program comprising program code means for causing a radiation-and-detection system as defined in claim 1 to carry out the steps of the method as claimed in claim 18, when the computer program is run on a computer controlling the radiation-and-detection system.

Preferred embodiments of the invention are defined in the dependent claims.

It shall be understood that the radiation-and-detection system of claim 1, the radiation-and-detection system of claim 13, the image generation system of claim 15, the imaging system of claim 17, the radiation-and-detection method of claim 18 and the computer program of claim 19 have similar and/or identical preferred embodiments as defined in the dependent claims.

Is shall be understood that preferred embodiments of the invention can also be combinations of, for example, two or more dependent claims with the respective independent claim.

### BRIEF DESCRIPTION OF THE DRAWINGS

These and other aspects of the invention will be apparent from and elucidated with reference to the embodiment(s) described hereinafter. In the following drawings

FIG. 1 shows schematically an embodiment of an radiation-and-detection system in accordance with the invention,

FIG. 2 shows schematically a radiation unit, a detection unit and a blocking element of the radiation-and-detection system in accordance with the invention,

FIG. 3 shows schematically an embodiment of an calibration and image generation device in accordance with the invention,

FIG. 4 shows a flowchart illustrating an embodiment of a method for calibrating a radiation-and-detection system in accordance with the invention and

FIG. 5 shows a flowchart illustrating an imaging method in accordance with the invention.

### DETAILED DESCRIPTION OF EMBODIMENTS

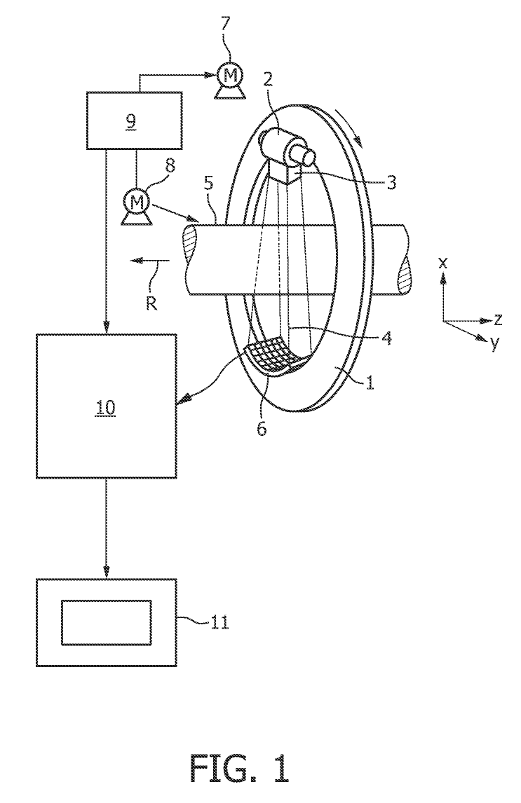

FIG. 1 shows a radiation-and-detection system, which is in this embodiment a CT system. The CT system includes a gantry 1, which is capable of rotation about an axis of rotation R which extends parallel to the z direction. The radiation unit 2, which is in this embodiment an X-ray stereo tube 2, is mounted on the gantry 1. The X-ray stereo tube is provided with a collimator device 3, which forms a conical radiation beam 4 (cone beam) from the radiation generated by the X-ray stereo tube 2. The radiation traverses an object (not shown), such as a patient, in a region of interest in a cylindrical examination zone 5. After having traversed the examination zone 5, the X-ray beam 4 is incident on an X-ray detection unit 6, which is a two-dimensional detector mounted on the gantry 1.

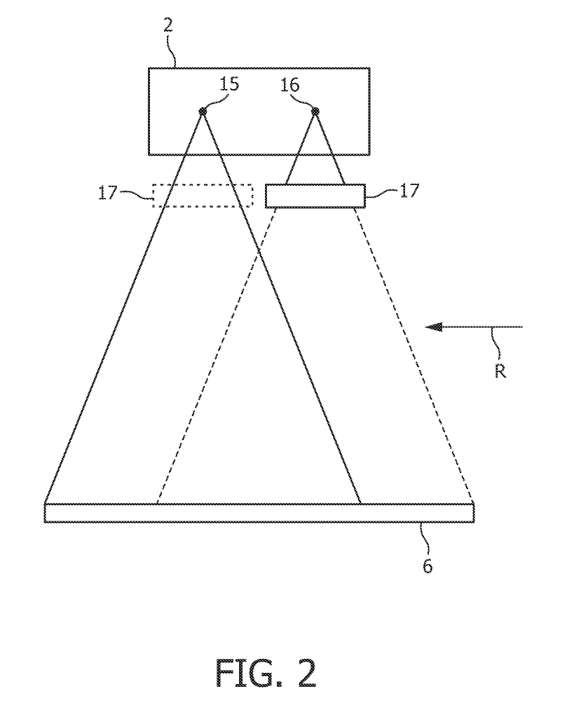

The X-ray stereo tube 2 comprises a first emanating region being a first focal spot 15 and a second emanating region being a second focal spot 16. The first focal spot 15 and the second focal spot 16 are located on a line parallel to the axis of rotation R and with an offset relative to each other, i.e. the first focal spot 15 and the second focal spot 16 are located with a distance between them on a line parallel to the axis of rotation R. The X-ray stereo tube 2 is schematically shown in FIG. 2.

The X-ray stereo tube 2 is adapted such that the region of interest is alternately illuminated by the radiation emanating from the first focal spot 15 and the second focal spot 16. It is preferred that the first focal spot 15 is pulsed periodically such that radiation emanates from the first focal spot 15 at predetermined periodic time points. It is also preferred that the second focal spot 16 is periodically pulsed such that the second focal spot 16 emanates radiation at predetermined periodical time points. The first focal spot 15 and the second focal 16 have preferentially the same periodic time, and the pulse period of the first focal spot is shifted by a half periodic time with respect to the pulse period of the second focal spot 16.

The collimator 3 comprises a blocking element 17, for example, a metal plate, which can be positioned at a first position in order to block the radiation emanating from the first focal spot 15 (shown in dashed lines in FIG. 2) and which can be positioned at a second position in order to block the radiation emanating from the second focal spot 16 (shown in solid lines in FIG. 2).

The gantry 1 is driven at a preferably constant but adjustable angular speed by a motor 7. A further motor 8 is provided for displacing the object, for example, a patient who is arranged on a patient table in the examination zone 5, parallel to the direction of the axis of ration R or of the z axis. These motors 7 or 8 are controlled by a control unit 9, for instance, such that the radiation unit 2 and the examination zone 5 move relative to each other along a helical trajectory. However, it is also possible that the object or the examination zone 5 is not moved, but that only the X-ray stereo tube 2 is rotated, i.e., that the X-ray stereo tube 2 and the examination zone 5 move relative to each other along a circular trajectory. The motors 7, 8, the gantry 1 and preferentially a patient table form moving unit, which allows the region of interest to be illuminated from different directions by the X-ray stereo tube 2.

The data acquired by the detection unit 6, which are detection values, are provided to an calibration and image generation device 10 for calibrating the CT system and for generating an image of the region of interest. The generated image can finally be provided to a display 11 for displaying the image. Also the calibration and image generation device 10 is preferably controlled by the control unit 9. Alternatively or in addition the calibration and image generation device 10 can comprise a control unit for controlling the calibration and image generation device 10 only.

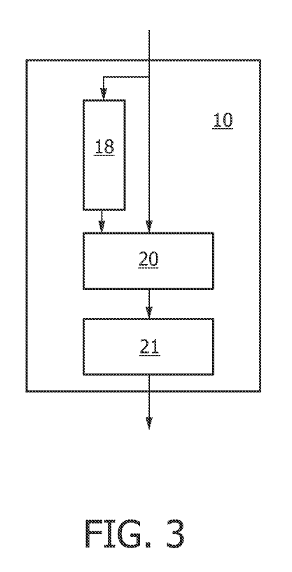

The calibration and image generation device 10 is schematically shown in FIG. 3 and comprises a calibration unit 18, a correction unit 20 and a reconstruction unit 21.

The CT system is adapted such that the first focal spot 15 emanates radiation at first time intervals, which are first time points in this embodiment, and that the second focal spot 16 emanates radiation at second time intervals, which are second time points in this embodiment. At the first time points only the first focal spot 15 is supposed to emanate radiation, but practically there will be some residual X-ray flux also from the second focal spot 16 at the first time points. Furthermore, at the second time points only the second focal spot 16 is supposed to emanate radiation, but practically also the first focal spot 15 emanates some residual X-ray flux at the second time points.

The detection unit 6 is adapted for detecting first detection values at the first time points and second detection values at the second time points.

The first detection values are influenced by the residual X-ray flux of the second focal spot 16 and by an afterglow of the detection unit 6, in particular, caused by the radiation of the second focal spot 16, which was emanated during the second time points, and by the residual X-ray flux of the second focal spot 16. The second detection values are influenced by the residual X-ray flux of the first focal spot 15 and by an afterglow of the detection unit 6, in particular, caused by the radiation of the first focal spot 15, which was emanated at the first time points, and by the residual X-ray flux of the first focal spot 15.

The imaging system can be operated in a calibration mode, in which calibration values are determined, and in an image generation mode, in which images are generated.

In the calibration mode, the first and second detection values received by the calibration and image generation device 10 are inputted to the calibration unit 18. In the image generation mode, the first and second detection values are directly inputted to the correction unit 20.

In the calibration mode, the calibration unit 18 determines calibration values for correcting the first detection values with respect to the influence of the radiation of the second focal spot 16 to the first detection values, wherein the calibration unit 18 is adapted for using at least one of the first and second detection values to determine calibration values, which will, in the image generation mode, be used by the correction unit 20 for correcting the first detection values.

The correction unit 20 is adapted for correcting the first and second detection values using the determined calibration values. This correction will be described in more detail further below.

The reconstruction unit 21 is adapted for reconstructing an image of the region of interest using the corrected first and second detection values. The reconstruction can be performed by using a backprojection method or by using another known reconstruction method.

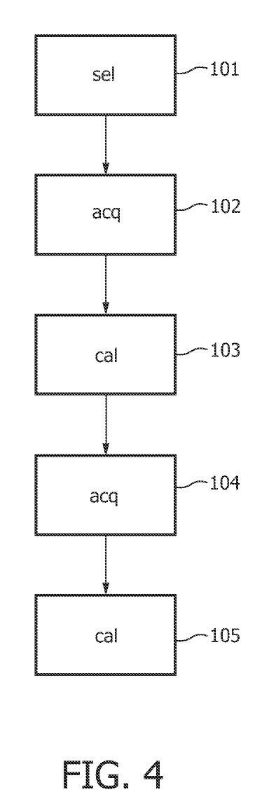

An embodiment of a method for calibrating the CT system in accordance with the invention will now be described in more detail with respect to a flowchart shown in FIG. 4.

In step 101 the calibration mode is selected. This selection can be performed automatically, for example, after a predetermined number of scans have been performed, or the imaging system can provide a graphical user interface, which allows a user to select the calibration mode.

In step 102 first and second detection values are acquired. In order to acquire the first and second detection values, the X-ray stereo tube 2 rotates around the region of interest, in which no object is present, and the region of interest is not moved, i.e. the X-ray stereo tube 2 travels along a circular trajectory around the region of interest. Alternatively, in another embodiment, the X-ray stereo tube 2 rotates around the region of interest, and the region of interest is moved parallel to the z direction, i.e. the X-ray stereo tube 2 travels along a helical trajectory. The region of interest can be moved by moving, for example, a patient table on which the object, for example, a patient, is located.

During acquisition in step 102, the blocking element 17, which is a metal plate in this embodiment, is located at the second position blocking the radiation emanating from the second focal spot 16. Thus, radiation emanates alternately and periodically from the first focal spot 15 and the second focal spot 16, but only the radiation emanating from the first focal spot 15 reaches the detection unit 6. The first focal spot 15 emanates radiation at the first time points, and the second focal spot 16 emanates radiation at the second time points. The detection unit 6 detects first detection values at the first time points and second detection values at the second time points.

In step 103 the calibration unit 18 determines calibration values for correcting second detection values by using the detected first and second detection values. For a better understanding of the determination of the calibration values this determination will be mathematically described in the following.

The intended output of the X-ray stereo tube 2 for the first focal spot 15 can be modeled as an infinite series of pulses:

$${S}_{1} ( t ) = {S}_{0} {\sum }_{i} δ ( t - i 2 Δ t ) . \qquad ( 1 )$$

The first time points are indicated by i2Δt with i=1, 2, 3 . . . . The δ function is defined by following equation:

$$δ ( x ) = \{ \begin{matrix}1 & for x = 0 \\ 0 & for x \neq 0.\end{matrix} \qquad ( 2 )$$

S₀ is the actual flux of the tube.

The second time points are indicated by (2i+1)Δt. The term Δt denotes the periodic time of the first focal spot 15 and the second focal spot 16. The part of the second detection values, which is caused by the afterglow of the detection unit 6 due to the radiation of the first focal spot 15 emanated at the first time points can be modeled by following equation:

$${A}_{0} = {S}_{0} {\sum }_{i = 0}^{∞} {e}^{- ( 2 i + 1 ) Δ t / {t}_{0}} = {S}_{0} \frac{{e}^{Δ t / {t}_{0}}}{{e}^{2 Δ t / {t}_{0}} - 1} . \qquad ( 3 )$$

The time constant t₀ of the afterglow of the detection unit 6 can be determined by known afterglow determination methods, for example, the afterglow determination method disclosed in Hsieh et al., “Investigation of a Solid-State Detector for Advanced Computed Tomography”, IEEE Transactions on Medical Imaging, vol. 19, no 9, 2000, pp 930-940.

The second detection values are further influenced by the residual X-ray flux of the first focal spot 15, i.e. the residual X-ray flux of the first focal spot 15 is directly detected and, causes, in addition, another afterglow contribution to the second detection values. This other afterglow contribution caused by the residual X-ray flux of the first focal spot 15 can be modeled by following equation:

$${A}_{1} = ɛ {S}_{0} {\sum }_{i = 0}^{∞} {e}^{- ( 2 i + 2 ) Δ t / {t}_{0}} = ɛ {S}_{0} \frac{1}{{e}^{2 Δ t / {t}_{0}} - 1} \qquad ( 4 )$$

In equation (4) εS₀ indicates the residual X-ray flux.

A second detection value acquired at the second time point (2i+1)Δt can now be modeled as the sum of all three contributions, i.e. the residual X-ray flux of the first focal spot 15, the afterglow caused by this residual X-ray flux and the afterglow caused by the radiation of the first focal spot 15 during the first time points:

$$\begin{matrix}M ( ( 2 i + 1 ) Δ t ) = {S}_{0} ( ɛ + \frac{{e}^{Δ t / {t}_{0}}}{{e}^{2 Δ t / {t}_{0}} - 1} + \frac{ɛ}{{e}^{2 Δ t / {t}_{0}} - 1} ) \\ = {S}_{0} ( \frac{{e}^{Δ t / {t}_{0}}}{{e}^{2 Δ t / {t}_{0}} - 1} + \frac{{ɛe}^{2 Δ t / {t}_{0}}}{{e}^{2 Δ t / {t}_{0}} - 1} ) \\ = {S}_{0} \frac{{e}^{Δ t / {t}_{0}} + {ɛe}^{2 Δ t / {t}_{0}}}{{e}^{2 Δ t / {t}_{0}} - 1} .\end{matrix} \qquad ( 5 )$$

In equation (5) M((2i+1)Δt) indicates the second detection value acquired at the second time point (2i+1)Δt.

In the same way, a first detection value M(2iΔt), which is acquired at the first time point 2iΔt, while the blocking element 17 is still located at the second position, can be modeled in accordance with following equation:

$$\begin{matrix}M ( 2 i Δ t ) = {S}_{0} ( 1 + \frac{1}{{e}^{2 Δ t / {t}_{0}} - 1} + \frac{{ɛe}^{Δ t / {t}_{0}}}{{e}^{2 Δ t / {t}_{0}} - 1} ) \\ = {S}_{0} \frac{{e}^{2 Δ t / {t}_{0}} + {ɛe}^{Δ t / {t}_{0}}}{{e}^{2 Δ t / {t}_{0}} - 1} .\end{matrix} \qquad ( 6 )$$

Dividing equation (6) by equation (5) yields:

$$\begin{matrix}R = \frac{M ( 2 i Δ t )}{M ( ( 2 i + 1 ) Δ t )} \\ = \frac{{e}^{2 Δ t / {t}_{0}} + {ɛe}^{Δ t / {t}_{0}}}{{e}^{Δ t / {t}_{0}} + {ɛe}^{2 Δ t / {t}_{0}}} \\ = \frac{{e}^{Δ t / {t}_{0}} + ɛ}{1 + {ɛe}^{Δ t / {t}_{0}}} ,\end{matrix} \qquad ( 7 )$$

which is equivalent to

$$ɛ = \frac{{e}^{Δ t / {t}_{0}} - R}{{e}^{Δ t / {t}_{0}} R - 1} . \qquad ( 8 )$$

Equation (8) allows to determine a calibration value ε, which is a measure for the residual X-ray flux. Since ε is a second calibration value, it will in the following be denoted by ε₂. If ε is determined by using first and second detection values, while the blocking element is located at the first position, the determined calibration value is a first calibration value and will therefore in the following be denoted by ε₁.

Thus, in order to determine the second calibration values ε₂, in step 102 first detection values M(2Δt) and second detection values M((2i+1)Δt) are acquired, while the blocking element 17 is located at the second position. In step 103 the calibration value ε₂ is determined by dividing subsequent measurements M(2Δt) and M(2i+1Δt) yielding the radio R and by calculating the calibration value ε₂ in accordance with equation (8).

In other embodiments, several ratios R can be calculated, an average R can be determined by averaging over the several calculated R, and this average R can be used for calculating the calibration value.

In step 104, the blocking element 17 is moved to the first position. The X-ray stereo tube 2 is activated, and radiation emanates alternately and periodically from the first focal spot 15 and the second focal spot 16, but only the radiation emanating from the second focal spot 16 reaches the detection unit 6. The first focal spot 15 emanates radiation at the first time points, and the second focal spot 16 emanates radiation at the second time points. The detection unit 6 detects first detection values at the first time points and second detection values at the second time points.

In step 105, the first calibration unit determines first calibration values ε₁ by dividing subsequent measurements M ((2i+1)Δt) and M(2Δt) yielding the ratio R and by calculating the calibration values ε in accordance with equation (8), wherein during calculating the first calibration value, in equation (8), ε has to be replaced by ε₁.

The calibration values ε₁ and ε₂ and t₀, which is the time constant of the afterglow of the detection unit, are preferentially stored in the memory of the calibration and image generation device 10, in order to provide these calibration values to the correction unit 20 for correcting detection values, which have been acquired in the image generation mode.

The correction of the first and second detection values during the image generation mode will now be described by using following mathematical derivation.

In the image generation mode, a certain detector pixel is supposed to measure the intensity of an X-ray beam passing through an object. The intensity now varies with time because different parts of the object are illuminated due to the rotation of the CT system. The line integral through the object at a sample point m of the detection unit 6 is indicated by Lₘ. The desired measurement, i.e. the expected measurement without afterglow and residual X-ray flux, can be modeled by following equation:

Sₘ=S₀e^{-L}^{ₘ}, (8)

wherein Sₘ denotes the detection value at sample point m. The actual measurement is, however, corrupted by afterglow and the residual X-ray flux. The relation between the measured corrupted detection values and the desired uncorrupted detection values can be modeled by following equation:

Ŝ=TUS, (9)

wherein Ŝ is a vector formed by all individual measured corrupted detection values Ŝₘ and wherein S indicates a vector formed by all desired uncorrupted detection values Sₘ. The matrix U is defined as follows:

$$U = ( \begin{matrix}1 & {ɛ}_{2} / 2 & 0 & 0 & 0 & … \\ {ɛ}_{1} / 2 & 1 & {ɛ}_{1} / 2 & 0 & 0 & … \\ 0 & {ɛ}_{2} / 2 & 1 & {ɛ}_{2} / 2 & 0 & … \\ 0 & 0 & {ɛ}_{1} / 2 & 1 & {ɛ}_{1} / 2 & ⋱ \\ 0 & 0 & 0 & {ɛ}_{2} / 2 & 1 & ⋱ \\ ⋮ & ⋮ & ⋮ & ⋱ & ⋱ & ⋱\end{matrix} ) . \qquad ( 10 )$$

Here, the contribution of the focal spot with the residual flux is approximated by the average intensity of the readings at the preceding and following sample points.

Furthermore, the matrix T is defined as follows:

$$T = ( \begin{matrix}1 & 0 & 0 & 0 & … \\ {e}^{- Δ t / {t}_{0}} & 1 & 0 & 0 & ⋱ \\ {e}^{- 2 Δ t / {t}_{0}} & {e}^{- Δ t / {t}_{0}} & 1 & 0 & ⋱ \\ {e}^{- 3 Δ t / {t}_{0}} & {e}^{- 2 Δ t / {t}_{0}} & {e}^{- Δ t / {t}_{0}} & 1 & ⋱ \\ ⋮ & ⋱ & ⋱ & ⋱ & ⋱\end{matrix} ) . \qquad ( 11 )$$

Thus, in order to correct the detection values, the calibration values t₀ and the calibration values ε₁ and ε₂ obtained by calibrating the CT system in accordance with the invention are used to form the matrixes T and U, and the desired uncorrupted corrected detection values S are determined by inverting equation (9). For inverting equation (9) known matrix inversion methods can be used. Thus, the first and second detection values are corrected by inverting equation (9).

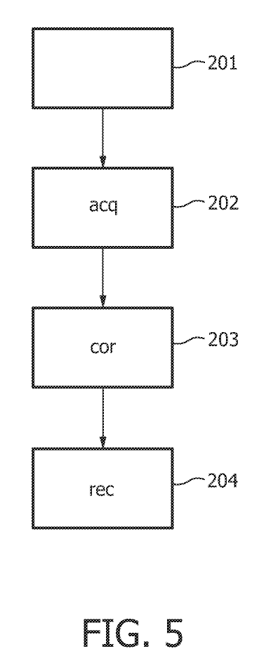

An image generation method for generating an image of the region of interest will now be described in more detail with reference to a flowchart shown in FIG. 5.

In step 201 the image generation mode is selected, for example, by asking a user, whether he wants to continue in the image generation mode or in the calibration mode, by using a graphical user interface. Preferentially, the image generation mode is the default mode, i.e., if a selection is not performed, the image generation mode is preferentially executed.

In step 202, an object is located within the region of interest and the X-ray stereo tube 2 travels along a circular or helical trajectory around the region of interest. The X-ray stereo tube 2 emanates radiation alternately and periodically from the first focal spot 15 and the second focal spot 16. The blocking element 17 is not placed in the propagation direction of the radiation, i.e. the blocking element 17 does not block the radiation, which emanates from the first focal spot 15 and the second focal spot 16 alternately. The detection unit 6 detects the radiation, which has passed the region of interest, and generates first and second detection values. The first focal spot 15 emanates the radiation at the first time points, and the second focal spot 16 emanates radiation at the second time points. The first detection values are acquired at the first time points, and the second detection values are acquired at the second time points. The acquired detection values are transmitted to the correction unit 20.

In step 203, the correction unit 20 corrects the first and second detection values using the stored calibration values ε₁, ε₂, t₀. The correction is performed in accordance with equation (9). Thus, the measured corrupted first and second detection values are arranged in a vector Ŝ, the matrix T and the matrix U are formed in accordance with equation (10) and (11) by using the stored calibration values, and the corrected detection values S are calculated by inverting equation (9) using known matrix inversion methods. Alternatively or in addition, the matrix T and the matrix U can be stored in a memory of the calibration and image generation unit 10, in order not to form the matrix T and the matrix U during each image generation process.

In step 204, an image of the region of interest is reconstructed using the corrected first and second detection values. For this reconstruction, well known reconstruction techniques can be used, for example, the filtered backprojection technique. An appropriate reconstruction technique is, for example, disclosed in U.S. Pat. No. 6,426,989

The invention is not limited to the above described embodiment. For example, a radiation-and-detection system in accordance with the invention can also be a combination of a pulsed X-ray tube having only one focal spot and a corresponding detection unit. In this case, the radiation-and-detection system shown in FIG. 1 comprises a radiation unit 2, which is the pulsed X-ray tube having now one focal spot, wherein, in a calibration mode, first detection values are acquired, while the X-ray tube emanates radiation, and second detection values are acquired, while the X-ray tube does not emanate or emanates only a residual X-ray flux. The calculation unit 18 is, in this case, adapted for determining calibration values by using successive first and second detection values as described above. In an image generation mode the radiation values can be used for correcting first and second detection values, for example, as also described above.

Furthermore, in accordance with the invention, the radiation unit can also comprise more than two emanating regions illuminating a detection unit alternately.

The invention is not limited to a radiation-and-detection system comprising an X-ray stereo tube. For example, in the above described embodiment, instead of one X-ray tube having two focal spots two X-ray tubes can be used having each one pulsed focal spot.

Other variations to the disclosed embodiments can be understood and effected by those skilled in the art and practicing the claimed invention, from a study of the drawings of the disclosure, and the appended claims.

While the invention has been illustrated and described in detail in the drawings and in the foregoing description, such illustration and description are to be considered illustrative or exemplary and not restrictive. The invention is not limited to the disclosed embodiments.

In the claims, the word “comprising” does not exclude other elements or steps, and the indefinite article “a” or “an” does not exclude a plurality.

A computer program may be stored/distributed on a suitable medium, such as an optical storage medium or a solid-state medium supplied together with or as part of other hardware, but may also be distributed in other forms, such as via the internet or other wired or wireless telecommunication systems.

Any reference signs in the claims should not be construed as limiting the scope.

1. A radiation-and-detection system comprising
a radiation unit having a first emanating region and a second emanating region for alternately illuminating the detection unit with radiation of the first emanating region at first time intervals and with radiation of the second emanating region at second time intervals,
a detection unit for detecting values depending on the radiation, the detection unit being adapted for detecting first detection values at the first time intervals and second detection values at the second time intervals,
a calibration unit
for calibrating the radiation-and-detection system with respect to the influence of the radiation of the second emanating region to the first detection values, the calibration unit being adapted for determining calibration values, which depend on the influence of the radiation of the second emanating region to the first detection values, for correcting the first detection values, and
for calibrating the radiation-and-detection system with respect to the influence of the radiation of the first emanating region to the second detection values, the calibration unit being adapted for determining calibration values, which depend on the influence of the radiation of the first emanating region to the second detection values, for correcting the second detection values.

2. The radiation-and-detection system as defined in claim 1, wherein the calibration unit is adapted for using at least one of the first and second detection values to determine the calibration values for correcting the first detection values, and
wherein the calibration unit is adapted for using at least one of the first and second detection values to determine the calibration values for correcting the second detection values.

3. The radiation-and-detection system as claimed in claim 1, wherein the radiation-and-detection system comprises a prevention unit for preventing the radiation of one of the first and the second emanating regions from being detected by the detection unit.

4. The radiation-and-detection system as claimed in claim 1, wherein the prevention unit is a blocking element for blocking radiation.

5. The radiation-and-detection unit as claimed in claim 3,
wherein, for determining calibrating values for correcting the first detection values,
the prevention unit is adapted for preventing the detection of the radiation of the first emanating region by the detection unit,
the detection unit is adapted for detecting first and second detection values, and
the calibration unit is adapted for determining calibration values for correcting the first detection values by using a ratio of successive first and second detection values, and
wherein, for determining calibrating values for correcting the second detection values,
the prevention unit is adapted for preventing the detection of the radiation of the second emanating region by the detection unit,
the detection unit is adapted for detecting first and second detection values, and
the calibration unit is adapted for determining calibration values for correcting the second detection values by using a ratio of successive first and second detection values.

6. The radiation-and-detection system as claimed in claim 1, wherein the calibration unit is adapted for determining calibration values which depend on an afterglow value of the detection unit.

7. The radiation-and-detection system as claimed in claim 1, wherein the calibration unit is adapted for determining calibration values which depend on residual radiation of an emanating region.

8. The radiation-and-detection system as claimed in claim 1, wherein the radiation-and-detection system comprises a correction unit being adapted for correcting the first and second detection values using the calibration values.

9. The radiation-and-detection system as claimed in claim 8, wherein the correction unit is adapted for correcting the first and second detection values using following steps:
modeling the first and second detection values as a matrix equation, wherein the first and second detection values are connected to corrected first and second detection values by a matrix, which depends on the calibration values,
recalculating the corrected first and second detection values by inverting the matrix equation.

10. The radiation-and-detection system as claimed in 8, wherein the radiation-and-detection system comprises a reconstruction unit for reconstructing an image of a region of interest located between the radiation unit and the detection unit using the corrected first and second detection values.

11. The radiation-and-detection system as claimed in claim 1,
wherein the radiation-and-detection system is a computed tomography system, and
wherein the radiation unit is an X-ray stereo tube.

12. A radiation-and-detection system comprising
a radiation unit having an emanating region for intermittently illuminating a detection unit at first time intervals,
a detection unit for detecting detection values depending on the radiation at second time intervals located between the first time intervals,
a calibration unit for calibrating the radiation-and-detection system with respect to the influence of the radiation emanated at the first time intervals to the detection values detected at the second time intervals, the calibration unit being adapted for determining calibration values, which depend on the influence of the radiation emanated at the first time intervals to the detection values detected at the second time intervals, for correcting the first detection values and second detection values.

13. The radiation-and-detection system as claimed in claim 12, wherein the detection unit is adapted for detecting first detection values at the first time intervals and second detection values at the second time intervals, and
wherein the calibration unit is adapted for using at least one of the first and second detection values to determine the calibration values for correcting the second detection values.

14. The radiation-and-detection system as claimed in claim 12, wherein the calibration unit is adapted for determining calibration values which depend on an afterglow value of the detection unit.

15. The radiation-and-detection system as claimed in claim 12, wherein the calibration unit is adapted for determining calibration values which depend on a residual radiation of the emanating region.

16. An image generation system for generating an image of a field of interest, the image generation system being provided with
first detection values being detected at first time intervals and second detection values being detected at second time intervals, wherein a radiation unit has alternately illuminated the detection unit with radiation from a first emanating region at first time intervals and a second emanating region at second time intervals,
calibration values, which depend on the influence of the radiation of the second emanating region to the first detection values, for correcting the first detection values,
calibration values, which depend on the influence of the radiation of the first emanating region to the second detection values, for correcting the second detection values,
the image generation unit comprising:
a correction unit being adapted for correcting the first and second detection values using the calibration values,
a reconstruction unit for reconstructing an image of a region of interest located between the radiation unit and the detection unit using the corrected first and second detection values.

17. A radiation-and-detection method for calibrating a radiation-and-detection system, wherein the radiation-and-detection method comprises following steps:
alternately illuminating a detection unit with radiation of a first emanating region at first time intervals and with radiation of a second emanating region at second time intervals,
detecting values depending on the radiation, wherein first detection values are detected at the first time intervals and second detection values are detected at the second time intervals,
calibrating the radiation-and-detection system with respect to the influence of the radiation of the second emanating region to the first detection values, wherein calibration values are determined, which depend on the influence of the radiation of the second emanating region to the first detection values, for correcting the first detection values, and
calibrating the radiation-and-detection system with respect to the influence of the radiation of the first emanating region to the second detection values, wherein calibration values are determined, which depend on the influence of the radiation of the first emanating region to the second detection values, for correcting the second detection values.

18. A computer program for calibrating a radiation-and-detection system, the computer program comprising program code means for causing a radiation-and-detection system as defined in claim 1, when the computer program is run on a computer controlling the radiation-and-detection system.
